(19) United States
(12) Patent Application Publication
(10) Pub. No.: US 2021/0241883 A1
(43) Pub. Date: Aug. 5, 2021

(54) MEDICAL IMAGE RECONSTRUCTION CLOUD SCHEDULER

(71) Applicant: KONINKLIJKE PHILIPS N.V., EINDHOVEN (NL)

(72) Inventors: THOMAS NETSCH, HAMBURG (DE); MICHAEL GÜNTER HELLE, HAMBURG (DE); THOMAS KOEHLER, NORDERSTEDT (DE); CLAAS BONTUS, HAMBURG (DE); CHRISTOPHE MICHAEL JEAN SCHÜLKE, HAMBURG (DE); TANJA NORDHOFF, HAMBURG (DE); DOUGLAS B. MCKNIGHT, CHARDON, OH (US)

(21) Appl. No.: 17/251,420

(22) PCT Filed: Jun. 17, 2019
(86) PCT No.: PCT/EP2019/065792

§ 371 Date: Dec. 11, 2020

Related U.S. Application Data
(60) Provisional application No. 62/686,743, filed on Jun. 19, 2018.

(51) Int. Cl. G16H 30/20; G16H 30/40; G06T 11/00

(52) U.S. Cl. CPC G16H 30/20; G16H 30/40; G06T 11/005; G06T 2207/10081; G06T 2207/10116; G06T 2207/10088; G06T 2207/10104; G06T 2207/10108

(57) ABSTRACT

A system (100) for reconstruction of medical images over a network comprises a scheduler (302) that schedules a reconstruction request (108) and the reconstruction request includes a medical image reconstruction of a subject according to an imaging protocol The scheduling includes scheduling of a plurality of events, each event with a corresponding time, and the plurality of events include at least one event with the corresponding time selected from a group consisting of a first time (520) to transmit raw image data (114) over a first network from a source node (116) to a reconstruction node (106), a second time (522) to reconstruct the medical image (118) by the reconstruction node, and a third time (524) to transmit the reconstructed medical image over a second network from the reconstruction node to a destination node (120).

### FIELD OF THE INVENTION

The following generally relates to medical imaging and medical informatics and more particularly to scheduling reconstruction of medical images and is described with particular application to Computed Tomography (CT), and is also amendable to Magnetic Resonance (MR), Positron Emission Tomography (PET), Single Proton Emission Computed Tomography (SPECT), Ultrasound (US), X-ray, and/or other imaging modality.

### BACKGROUND OF THE INVENTION

An imaging protocol specifies scanner configurations for data acquisitions that generate raw image data, types of image reconstructions to be performed, and can include other information, such as patient positioning, contrast administration instructions and image destination information. For example, data acquisition by the configured scanner generates raw data of a scanned subject, such k-space data in MR data acquisitions and projection data in CT acquisitions. Computers, typically part of the scanner hardware, reconstruct images from the generated raw image data.

A healthcare practitioner, such as a technician, positions the patient, and operates the scanner to scan the patient. The healthcare practitioner typically receives at least some reconstructed images, often in real time, which allows confirmation that the correct anatomy is included in the generated raw image data. After the scanning is completed and the anatomy confirmed, the patient is released. Additional reconstructions can proceed after the patient is released.

In some instances, imaging protocols include more advanced, additional reconstructions of the generated raw image data. The advanced reconstructions, such as iterative reconstructions, can improve resolution, provide more detailed analysis, such as material basis, remove artifacts, compensate for motion, and combinations thereof. These advanced reconstructions can significantly increase the computational requirements for the reconstructed images, such as increasing the reconstruction times from on the order of seconds to on the order of hours, and/or increasing the number and speed of computer processors or hardware units to perform the reconstructions.

One approach to mitigating costs of the increased computer hardware for each medical imaging scanner is to centralize reconstruction hardware (e.g. computer processors) from multiple medical imaging scanners that perform the advanced reconstructions. However, such centralized arrangement of reconstruction hardware is typically scheduled and optimized based on throughput by the reconstruction hardware, and do not consider other infrastructure limitations that can created further delays in timely delivery of reconstructed images for use in delivery of healthcare, such a reading by a diagnostic healthcare practitioner, planning a therapy treatment, delivering a treatment, diagnosing a patient at a follow-up appointment, and the like.

### SUMMARY OF THE INVENTION

Aspects described herein address the above-referenced problems and others. The following describes an approach for scheduling reconstruction of medical images over a network, e.g. computing resources in a cloud. The scheduling includes one or more of times for transmission of generated raw image data (e.g. projection data generated by detectors of a CT scanner) from a source node (e.g. a CT imaging system or scanner) to a reconstruction node (e.g. medical image reconstruction units) over a network, reconstruction of the generated raw image data into medical images at the reconstruction node, and transmission of reconstructed medical images from the reconstruction node to a destination node (e.g. an image storage subsystem) over the network. The scheduling can optimize according to an expected time, such as a start read time by a diagnostic healthcare practitioner, start time for planning a patient therapy, start time of diagnosing a patient condition, or other event subsequent to reconstruction. The cloud can include a plurality of reconstruction nodes and/or scheduling nodes. In some embodiments, the scheduling can include a forecast from a scheduling node in response to a reconstruction request.

In one aspect, a system for reconstruction of medical images over a network comprises a scheduler that schedules a reconstruction request and the reconstruction request includes a medical image reconstruction of a subject according to an imaging protocol. The scheduling comprises scheduling of a plurality of events, each event with a corresponding time, and the plurality of events include at least one event with the corresponding time selected from a group consisting of a first time to transmit raw image data over a first network from a source node to a reconstruction node, a second time to reconstruct the one or more medical images by the reconstruction node, and a third time to transmit the reconstructed the reconstructed medical image over a second network from the reconstruction node to a destination node.

In another aspect, a method for reconstruction of medical images over a network includes scheduling a reconstruction request for a medical image reconstruction of a subject according to an imaging protocol. The scheduling comprises scheduling of a plurality of events, each event with a corresponding time, and the plurality of events include at least one event with the corresponding time selected from a group consisting of a first time to transmit raw image data over a first network from a source node to a reconstruction node, a second time to reconstruct the medical image by the reconstruction node, and a third time to transmit the reconstructed medical image over a second network from the reconstruction node to a destination node.

In another aspect, a computer-readable storage medium carrying instructions controls one or more processors to schedule a reconstruction request for a medical image reconstruction of a subject according to an imaging protocol. The scheduling comprises scheduling of a plurality of events, each event with a corresponding time, and the plurality of events include at least one event with the corresponding time selected from a group consisting of a first time to transmit raw image data over a first network from a source node to a reconstruction node, a second time to reconstruct the medical image by the reconstruction node, and a third time to transmit the reconstructed medical image over a second network from the reconstruction node to a destination node.

In another aspect, a system for reconstruction of medical images over a network includes a scheduler that schedules a medical imaging examination of a subject with a plurality of events subsequent to the medical imaging examination according to a delivery time. The medical imaging examination of the subject generates raw image data. At least one event of the plurality of events includes a reconstruction of the raw image data into a reconstructed medical image.

In another aspect, a system for delivery of radiological reports includes a computer processor configured to schedule a plurality of readers of reconstructed medical images and delivery of radiological reports from reading the reconstructed medical images according to a schedule of a plurality of medical imaging examinations. Each reader of the plurality of readers is matched to one of the plurality of scheduled medical imaging examinations based on an imaging protocol and an expected delivery time of a reconstructed medical image to the reader.

These and other aspects of the invention will be apparent from and elucidated with reference to the embodiment(s) described hereinafter.

### BRIEF DESCRIPTION OF THE DRAWINGS

The invention may take form in various components and arrangements of components, and in various steps and arrangements of steps. The drawings are only for purposes of illustrating the preferred embodiments and are not to be construed as limiting the invention.

FIG. 1 schematically illustrates an embodiment of a system for scheduling reconstruction of medical images in a cloud.

FIG. 2 schematically illustrates an example topological arrangement of nodes in the cloud.

FIG. 3 schematically illustrates an embodiment of a combined scheduling and reconstruction node.

FIG. 4 schematically illustrates an embodiment of a reconstruction requesting node.

FIG. 5 schematically illustrates an embodiment of static scheduling.

FIG. 6 schematically illustrates an embodiment of dynamic scheduling.

FIG. 7 schematically illustrates another embodiment of static scheduling.

FIG. 8 schematically illustrates another embodiment of static scheduling.

FIG. 9 illustrates a flowchart in accordance with an embodiment(s) herein.

FIG. 10 illustrates a flowchart in accordance with an embodiment(s) herein.

FIG. 11 illustrates a flowchart in accordance with an embodiment(s) herein.

FIG. 12 depicts an example dashboard display.

### DETAILED DESCRIPTION OF EMBODIMENTS

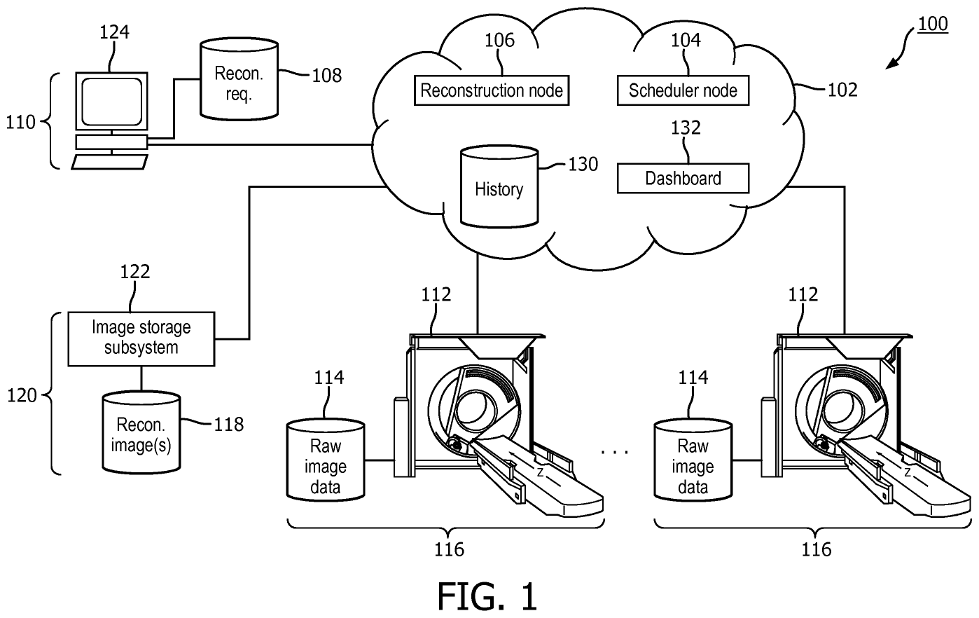

With reference to FIG. 1, an embodiment of a system 100 for scheduling reconstruction of medical images over a network 102 is schematically illustrated. The network 102 includes a communication network for transmitting analog and/or digital data, which can include wired connections, wireless connections and combinations thereof; data (e.g., broadband, Wi-Fi) networks, cellular networks, and combinations thereof; public networks, private networks, and combinations thereof; electrical digital data transmission, which can include radio frequency (RF) digital data transmission, optical digital data transmission, wired digital data transmission, etc., and combinations thereof. Lines between components in the diagram of FIG. 1 indicate communications paths.

The system 100 includes a scheduling node 104 and reconstruction node 106, which are accessible via the network 102. The scheduling node 104 receives a reconstruction request 108 from a reconstruction requesting node 110 according to an imaging protocol. In some embodiments, the reconstruction request 108 can include a work-list, such as a work-list generated daily and downloaded to each medical imaging scanner 112 that provides a schedule of imaging examinations and identifies and/or includes the imaging protocol. In some embodiments, the reconstruction request 108 includes an imaging examination schedule request according to the imaging protocol.

The scheduling node 104 schedules the reconstruction request for one or more medical image reconstructions of a subject according to the imaging protocol, e.g., based on at least the information in the imaging protocol. The scheduling includes a first time to transmit raw image data 114 over a first network from a source node 116 to the reconstruction node 106, a second time to reconstruct the medical image(s) 118 by the reconstruction node 106, and a third time to transmit the reconstructed medical image 118 over a second network from the reconstruction node 106 to a destination node 120. The first and second networks can be the same or different. In some embodiments, the reconstruction requesting node 110 initiates the reconstruction request. For example, a patient is to be scheduled for an imaging examination at a date and time X and with a specified imaging protocol A, and in response to scheduling the patient for the imaging examination, a reconstruction request 108 is initiated by the reconstruction requesting node 110 and sent to the scheduling node 104. In some embodiments, the scheduling node 104 retrieves the reconstruction request 108, such as by polling of worklists for each medical imaging scanner 112.

In some instances, the scheduling provides advantages over conventional practice, which does not consider other resources involved in delivery of reconstructed images to a reading radiologist. For example, conventional practice does not consider other resources in addition to reconstruction processors in separating the reconstruction processors from the medical imaging scanner, such as bandwidth for transmitting the raw image data 114 and bandwidth for transmitting the reconstructed medical image 118. The bandwidths can be different according to network paths, and in some instances dynamic according to load at time of transmission.

The source node 116 and the destination node 120 can be the same or different. Each of the source node 116 and the destination node 120 can include the medical imaging scanner 112, an image storage subsystem 122, network attached storage, a workstation, a server and combinations thereof. The medical imaging scanner 112 generates the raw image data 122 with medical imaging modalities that include Computed Tomography (CT), Magnetic Resonance (MR), Positron Emission Tomography (PET), Single Proton Emission Computed Tomography (SPECT), Ultrasound (US), and combinations thereof. The medical imaging scanner 112 can include components, such as a console, locally connected workstation, and the like. Examples of the image storage subsystem 122 include a Picture Archiving and Communication System (PACS), Radiology Information (RIS), Hospital Information System (HIS), Electronic Medical Record (EMR), and the like. For example, a source node 116 and the destination node 120 can both be the image storage subsystem 122, where an organization stores all image data on a central system including the raw image data 116 and the reconstructed medical image 118. In another example, the source node 116 includes the medical imaging scanner 112, and the destination node 120 includes the image storage subsystem 122.

The reconstruction requesting node 110 can include the medical imaging scanner 112, the image storage subsystem 122, another computer server or system, and/or a workstation 124, such as used for scheduling an imaging examination for a patient. Combinations are contemplated. The reconstruction requesting node 110 can include either one or both of the source node 116 and the destination node 120.

The reconstruction request 108 includes or identifies the imaging protocol. The following information is mapped from the included or identified imaging protocol: an estimated quantity of raw image data generated during the imaging examination, a type and parameters for the reconstruction to be performed, and an estimated quantity of the image data or an estimated size of the medical image generated by the reconstruction. For example, consider a reconstruction request that identifies a coronary CT angiography protocol that requires motion compensated reconstruction. In one example, this is mapped to 6.4 gigabytes (GB) of projection data, and an iterative reconstruction algorithm with compensated motion and gated cardiac motion phases, which maps to 5.8 GB of four dimensional (4D) reconstructed image data. In some embodiments, finer delineations are included, such as a number of files in the raw image data, estimated individual file sizes in the raw image data, a number of files in the reconstructed image data, and estimated individual file sizes. The mappings can be determined based on specifications of each medical imaging scanner 112, prior imaging examinations, and combinations thereof.

The reconstruction request 108 identifies the source node 116 for the raw image data 114 and the destination node 120 for the reconstructed medical image 118. In some embodiments, identifiers of the source node 116 and the destination node 120 are included in the imaging protocol or can mapped from the imaging protocol. In some embodiments, identifiers of the source node 116 and the destination node 120 default to the reconstruction requesting node 110. The reconstruction request 108 can include a static reconstruction request or a dynamic reconstruction request.

The system 100 can include a history 130 of executed schedule requests. The history 130 provides a record of performance for each event included in the schedule request. For example, transactional history of transmission, reconstruction, post processing events, reading, therapy planning events, and the like can be recorded in the history 130. The transaction history can include time stamps, identifiers of resources used in performing the event. For example, a transmission history can include an elapse time of transmission between two locations and the network path utilized. A reconstruction history can include an elapsed time of reconstruction, central processing unit (CPU) resources used, memory used, number of iterations of an iterative reconstruction, etc.

The history 130 can be distributed and stored locally as a collection of files across the various nodes. The history 130 can be centrally collected and stored as events are performed, such as recording messages sent by each node to a central collection. Combinations of storage arrangements are contemplated. The history 130 is suitably embodied by computer storage media and can include file organization, and database organization.

A dashboard 132 can provide analysis of the system and configured the system 100, such as a topological arrangement and scheduling parameters of the scheduler node 104, the reconstruction node 106, and a destination node. The dashboard 132 can provide an overview of the system 100 configuration and operation, resources utilization for different scheduling events, simulations of potential re-configurations, and can apply modifications to the configuration of the system 100.

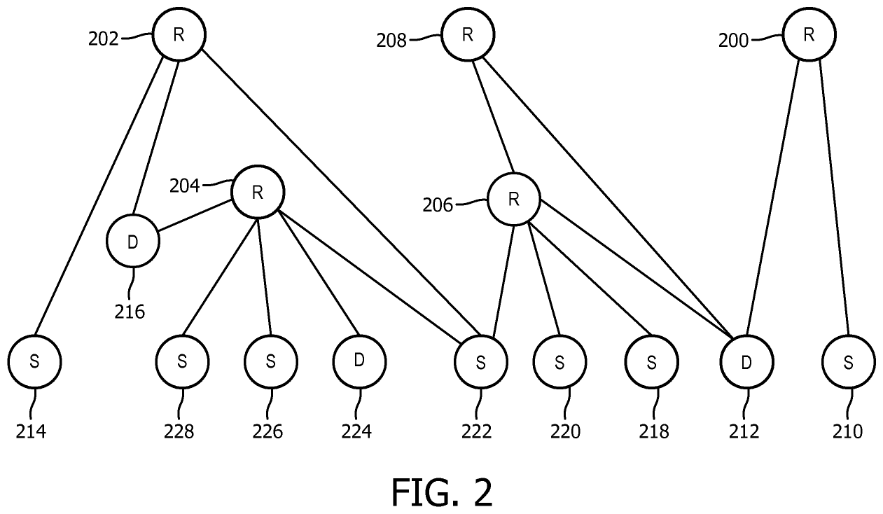

With reference to FIG. 2, an example topological arrangement of nodes in the network 102 is schematically illustrated. Nodes are indicated by circles, and communication paths of the network that connect the nodes are indicated by lines. In the example, the scheduling requestor nodes 110 include the source nodes 116 and are indicated by the letter “S” or S-node. In the example, destination nodes 120 are indicated by the letter “D” or D-node, and reconstruction nodes 106 include a scheduling node 104 are indicated by the letter “R” or R-node. The scheduling nodes 104 can operate separately and cooperatively, can operate independently from each other, and can operate in combinations thereof.

The reconstruction requests 108 can include a delivery time (e.g., a time to read the reconstructed image by a clinician such as a radiologist, a time for diagnosis, a time for planning a therapy, a time for a treatment), which is a specific time that completes one or more events subsequent to the reconstruction of the reconstructed medical image 118. For example, one event subsequent to the reconstruction of the reconstructed medical image 118 includes the transfer of the reconstructed medical image 118 to the destination node 120. Other examples of events include post processing of the reconstructed medical image 118, such as view generation, flypath generation, segmentation of anatomical structures, fitting anatomical or systemic models to the reconstructed medical image data 118, generation of parametric maps, quantitative measures of metabolic or anatomic features, identification and processing of relevant prior medical reports for a same patient or for a like patient population; and therapy planning, such as radiation therapy planning. In some embodiments, the time for diagnosis includes a follow-up schedule appointment for the patient depicted in the reconstructed medical image 118.

Responses to the reconstruction request 108 by the scheduling node 104 can include a confirmation of the reconstruction request 108, a forecast of the delivery time, such as the completed transfer of the reconstructed medical image 118 to the destination node 120, a notice that the scheduling node 104 is unable to schedule the reconstruction request 108 according to the delivery time, and combinations thereof. For example, the scheduling node 104 can return the notice and the forecast, which indicates the time to read cannot be satisfied, but the forecast indicates when delivery can be completed.

R-nodes 200, 202 and 204 illustrate independent operation of scheduling nodes 104, and R-nodes 206, 208 illustrate separate and cooperative operation of the scheduling nodes 104. An S-node 210 makes reconstruction requests of the R-node 200 and the reconstructed medical image 118 is to be returned to a D-node 212. The R-node 200 operates independently of other R-nodes. An S-node 216 makes reconstruction requests 108 of the R-node 202 and/or the R-node 204, which operate independently of each other. For example, the reconstruction request 108 is sent to the R-node 202 and the R-node 202 indicates in response that it cannot satisfy the request. The S-node 216 then sends the reconstruction request 108 to the R-node 204, which responds with a confirmation. In some embodiments, the reconstruction requests 108 can be sent to a plurality of R-nodes with the forecast returned by each R-node, and then the S-node selects a best forecast and responds to confirm the reconstruction request 108 with the selected R-node.

An S-node 218 and an S-node 220 make reconstruction requests 108 of the R-node 206. The reconstruction requests 108 that cannot be satisfied by the R-node 206 can be separately and cooperatively referred to the R-node 208. The referral can be visible or non-visible to the requesting S-node 218, 220. For example, a response to the reconstruction request 108 can include confirmation that the R-node 206 or any other cooperatively operated R-node, such as the R-node 208, will satisfy the request, or the response can include an identity of the particular R-node that will satisfy the request. Note that the S-nodes 210, 218, 220, such as three scanning locations can share the same D-node 212, such as a same PACS.

An S-node 222 makes reconstruction requests 108 of the R-nodes 202, 204, 206, which includes a combination of independent R-nodes, and separate and cooperative R-nodes. The reconstruction request 108 from the S-node 222 can include a selection of D-nodes 212, 216, and 224 for delivery of the reconstructed medical image 118, which can be dependent upon the selection of the R-node.

The R-node 204 illustrates an arrangement for receiving reconstruction requests 108 and/or the raw image data 114 from S-nodes 214, 222, 224, 226, and 228 (e.g. a plurality of source nodes 116, a plurality of scheduling nodes 104, and combinations thereof), and sending to D-nodes 216, 224 (a choice of destination node 120).

The topological arrangement illustrates an example tiered approach to cloud reconstruction. For example, a bottom row of S-nodes and D-nodes can be indicative of individual sites within a hospital or internal network, and a next row of R-nodes indicative of dedicated or owned hospital nodes accessible via an internal network portion of the network 102. A top-level row can be indicative of R-nodes accessible via an external network portion of the network 102, such as the Internet. The top-level R-nodes can be provided as a shared service among several healthcare organizations. Various configurations can be illustrated, such as a remote scanning location of a healthcare organization represented by the S-node 210 and an external service provider represented by the R-node 200.

The topological arrangement provides for flexibility and more deterministic scheduling over conventional practices, which do not balance reconstruction resources and network resources, account for dynamic changes in network resources, such as varying bandwidth, allow for growth with additional nodes or dynamic rearrangement of nodes, and provide for purchased dedicated hardware balanced with fee for service options.

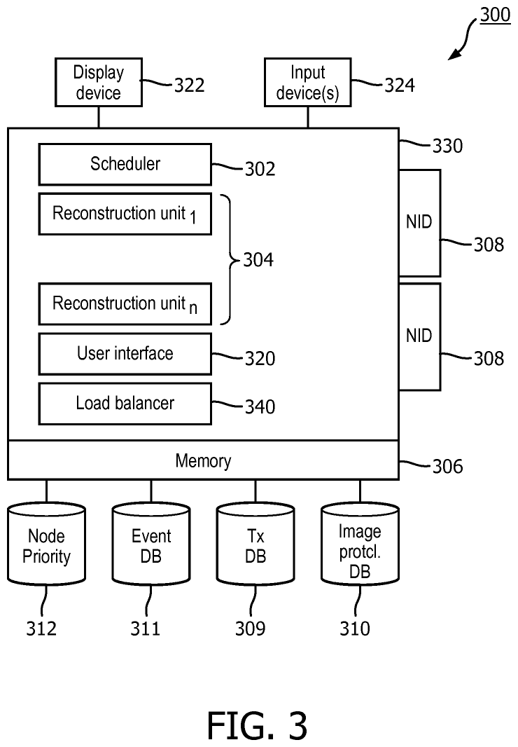

With reference to FIG. 3, an embodiment of a combined scheduling and reconstruction node 300 is schematically illustrated. The combined scheduling and reconstruction node 300 and each scheduling node 104 include a scheduler 302. The combined scheduling and reconstruction node 300 and each reconstruction node 106 include one or more reconstruction units 304. The combined scheduling and reconstruction node 300, the scheduling node 104, and the reconstruction node 106 each include electronic memory 306 and one or more network interface devices (“NIDs”) 308, such as network interface cards, network interface controller, network adapter, local area network (LAN) adapter, and the like.

The scheduler 302 can access an imaging protocol database 310, a transmission resource database 309, an event database 311, and combinations thereof. The databases 309, 310, and 311 are described individually, and in some embodiments can be combined. The imaging protocol database 310 maps imaging protocols to resource requirements, such as the estimated quantity of raw imaging data 114 to be transmitted, the type of reconstruction, and the estimated size of the reconstructed medical image 118 to be transmitted. The imaging protocol database 310 can include estimated duration of imaging examinations, such as the time from the start of the imaging examination to a time of the generation of raw image data 114. The estimates can be based on analysis of the history 130, such as linear regression analysis, averages, maximum, minimums, variances, combinations thereof, and the like. The estimates can include different levels of service or queues based on according to a delivery priority.

The imaging protocol database 310 can include reconstruction timing and duration estimates based on hardware resources of the reconstruction units 304 and modeling of transaction data from the history 130. Similarly, the estimates of reconstruction can be based on analysis of the history 130, such as linear regression analysis, averages, maximum, minimums, variances, combinations thereof, and the like. The estimates of reconstruction can include different levels of service or queues based on according to the delivery priority.

The transmission resource database 309 maps data quantities over different network paths between nodes to transmission time estimates according to bandwidth estimates or models for different network segments between nodes. The bandwidth estimates or models can be based on modeling of network traffic, i.e. day and time dependent from the history 130. For example, an estimated transmission time for the estimated quantity of the raw imaging data 114 and a time after generation of the raw imaging data 114 can be estimated. A model can include estimates according to polynomial curves fit to sampled periods of daily patterns (e.g. by minute or by hour) by network segment to predict variable transmission rates. Separate curves can be fit to weekend and holiday transmission rates.

The event database 311 maps other events to be completed prior to the delivery time after reconstruction of the reconstructed medical image 118 to time estimates for scheduling, such as post processing of the reconstructed medical image 118, and therapy planning. For example, an imaging protocol may be directed to a virtual colonoscopy, which includes post processing of anatomically segmenting the colon in the reconstructed medical image data 118, and constructing a flypath through the segmented colon. Estimates of the segmentation, and construction of the flypath can be included in the event database 311 for a time to read. In another example, the reading time and report generation can be included as another event in a time to delivery of a diagnosis. In another example, a therapy planning event includes receives the reconstructed medical image 118 at a destination node of a system for simulating radiation therapies, such as IMRT, and a time of delivery of a proposed treatment based the reconstructed medical image 118 is estimated. The example can be extended to include scheduling of the device to deliver the treatment as a time to delivery with N preceding events. The mapping can include different levels of service or queues based on according to the delivery priority.

Thus, from a proposed imaging examination appointment time and a proposed imaging protocol, estimates can be determined for timing and resource requirements of transmission of the raw image data 114 from the source node 116 to the reconstruction node 106, timing and duration of reconstruction at the reconstruction node 106, and timing and duration of transmission of the reconstructed medical image data 118 from the reconstruction node 106 to the destination node 120 can be determined.

The scheduler 302 can schedule with a variety of scheduling algorithms, such first come, first served, earliest deadline first, shortest remaining time first, fixed priority preemptive scheduling, round robin, multi-level queue scheduling, flow shop scheduling, manual scheduling, and combinations thereof. In some embodiments, the scheduler 302 includes a deep learning algorithm trained from the history 130. The scheduling encompasses the reconstruction units 304 performing the reconstruction, and the one or more NIDs 308 transmitting data, and can include other events after reconstruction. For example, a reading event that occurs after reconstruction can be scheduled with the scheduling of the imaging examination, such as in response to an order for the imaging procedure, and with the reconstruction. In another example, the reading event can be scheduled during or following the imaging examination, such as in response to an imaging examination of a trauma patient.

The scheduler 302 can include a scheduling node priority list 312, which is used to identify other scheduling nodes 104 that operate separately and cooperatively, i.e. as referrals. The scheduling node priority list 312 can include one or more scheduling nodes 104. The scheduling node priority list 312 can include an order of priority for selection of the scheduling node 104. The scheduling node priority list 312 can include a group of scheduling nodes 104 that are unordered and queried for fastest delivery according to the forecast.

The node priority list 312 can include an indicator whether the selected referrals are indicated in responses to the reconstruction requesting node 110. That is, the referral or selected scheduling node can be indicated and visible in addition to or instead of the scheduling node originally receiving the request and including the scheduling node priority list 312 to the reconstruction requesting node 110, or the selected scheduling node is not indicated and only the receiving scheduling node that originally receives the reconstruction request is visible to the reconstruction requesting node 110.

The combined scheduling and reconstruction node 300, the scheduling node 104, the dashboard 132, and the reconstruction node 106 each can include a user interface 320. The user interface 320 can operate a display device 322 to display scheduling status, such as a dashboard display, and operate one or more input device(s) 324 to manually schedule the reconstruction requests, alter the scheduling node priority list 312, modify the imaging protocol database 310, and combinations thereof. The user interface 320 is suitably embodied by a configured processor 330, the display device 322, and the input device(s) 324, and the configured processor 330 is configured to operate the display device 322 and the input device 324.

The configured computer processor 330 executes at least one computer readable instruction stored in the computer readable storage medium 306 (which excludes transitory medium), such as an optical disk, a magnetic disk, semiconductor memory of a computing device with the configured processor, and/or other non-transitory medium to perform the disclosed techniques. The configured processor 330 may also execute one or more computer readable instructions carried by a carrier wave, a signal or other transitory medium. The configured processor comprises a microprocessor, digital processor, optical processor, multi-core processor, multiple central processing units (CPUs), cluster arrange of processors, field programmable gate array (FPGA), graphic processing units (GPUs), combinations thereof, and the like.

The combined scheduling and reconstruction node 300, and the reconstruction node 106 each can include a load balancer 340, which supervises and balances transmissions, and balances work load between individual units of the reconstruction units 304. The load balancer 340 can limit the number of simultaneous transmissions via the NIDs 308 or selection of the NID 308 for a particular transmission, such as separate pipelines for transmission. The load balancer 340 can change scheduling algorithms or priority order, such as in response to a higher priority request, changed network conditions, reconstruction unit 304 availability, and combinations thereof. For example, the load balancer 340 changes the transmission of raw image data 114 from a selected source node 116 to a higher priority in response to an earlier than anticipated availability of the reconstruction unit 304, which makes the raw image data 114 available for processing by the earlier than anticipated available reconstruction unit 304. The load balancer 340 can assign reconstructions to individual reconstruction units 304 according to schedule by the scheduler 302, or modify the schedule in response to resource availability or non-availability.

The scheduler 302, the reconstruction units 304, the dashboard 132, and the load balancer 340 are suitably embodied by the configured processor 330 to perform the respective functions. The scheduling node priority list 312, the event database 311, the transmission resource database 309, and the imaging protocol database 310 are suitably embodied by electronic memory. The electronic memory can include data structures, file structures, and the like.

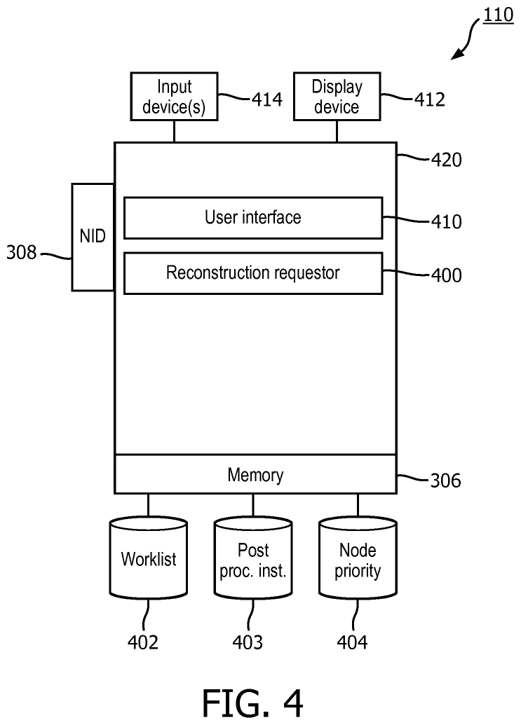

FIG. 4 schematically illustrates an embodiment of the reconstruction requesting node 110. The reconstruction requesting node 110 includes a reconstruction requestor 400. In some embodiments, the reconstruction requestor 400 provides access to and transmission of a locally stored work-list 402. In some embodiments, the reconstruction requestor 400 constructs and sends the reconstruction request 108 in response to an imaging examination appointment.

The reconstruction requesting node 110 can include a scheduling node priority 404 for determining the scheduling nodes 104 to receive the reconstruction request 108. The scheduling node priority 404 can include one or more scheduling nodes 104. The scheduling node priority 404 can be ordered, such as a first available scheduling node that can satisfy reconstruction request 108. For example, the scheduling node priority 404 includes three scheduling nodes 104, {A, B, C}. The reconstruction request 108 is submitted first to scheduling node A, and if scheduling node A is unable to satisfy the request, then the reconstruction request 108 is submitted to scheduling node B. If scheduling node B is unable to satisfy the reconstruction request 108, then the reconstruction request 108 is submitted to scheduling node C.

The scheduling node priority 404 can be unordered, and the reconstruction requesting node 110 selects the scheduling node 110 based on returned forecasts. For example, the node priority 404 includes two scheduling nodes 104, {D, E}. The reconstruction requesting node 110 submits the reconstruction request 108 to each of scheduling node D and scheduling node E. Each of scheduling nodes D and E returns a forecast that includes an estimated completion of delivery of the reconstructed medical image 118 to the destination node 120. The reconstruction requesting node 110 selects from the returned forecasts the corresponding scheduling node that provides the earliest delivery of the reconstructed medical image 118 to the destination node 120.

In some embodiments, the scheduling node priority 404 can be combinations of ordered and unordered nodes. For example, the scheduling node priority 404 includes {ordered A, B, {unordered C, D, B} }, where the first list is ordered and the second subset is unordered. The reconstruction requesting node 110 makes reconstruction requests 108 of scheduling nodes A and B in order. If neither of scheduling nodes A and B can satisfy the reconstruction request 108, then the reconstruction requestor node 110 selects among the best forecast of scheduling nodes C, D and B. Scheduling node B participates twice, first whether scheduling node B can satisfy the time to read, and second competing among scheduling nodes C and D for the best forecast, when the time to read cannot be satisfied.

The reconstruction requestor node 110 can include a user interface 410, which operates a display device 412 and input device(s) 414 to enter or modify the scheduling node priority 404, and to construct and/or send the reconstruction request 108. For example, the user interface 420 can be used to identify the worklist 402, an imaging protocol, the imaging examination schedule, files to be transferred, network address of the source node 116, network address of the destination node 120, etc.

The reconstruction requestor node 110 can include post processing instructions 403 based on the imaging protocol, the worklist 402, or provided separately. For example, a virtual colonoscopy imaging examination is scheduled, which indicates post processing events of colon segmentation from the reconstructed medical image 118, flypath determination through the segmented colon, and reading along the determined flypath by a radiologist qualified for reading virtual colonoscopies.

The user interface 410 is suitably embodied by a configured processor 420, the display device 412, and the input device(s) 414, and the configured processor 420 is configured to operate the display device 412 and the input device 414.

The configured computer processor 420 executes at least one computer readable instruction stored in the computer readable storage medium 306 (which excludes transitory medium), such as an optical disk, a magnetic disk, semiconductor memory of a computing device with the configured processor, and/or other non-transitory medium to perform the disclosed techniques. The configured processor 420 may also execute one or more computer readable instructions carried by a carrier wave, a signal or other transitory medium. The configured processor comprises a microprocessor, digital processor, optical processor, multi-core processor, multiple central processing units (CPUs), cluster arrange of processors, field programmable gate array (FPGA), graphic processing units (GPUs), combinations thereof, and the like. In some embodiments, the computer readable instructions are located locally, such as a dedicated “app”. In some embodiments, the computer readable instructions are located on a server, such as a hypertext transfer protocol (HTTP) server, and are delivered to the processor 420.

The scheduling node priority 404 is suitably embodied by electronic memory. The electronic memory can include data structures, file structures, and the like. In some embodiments, the scheduling node priority list 312 and the scheduling node priority 404 include a same format and structure.

In some embodiments, the scheduling node priority list 312 and the scheduling node priority 404 can be used in combination. For example, consider a hospital imaging location (e.g. source node) that one of several of a large hospital (e.g. plurality of source nodes), which is part of an overall healthcare network. The hospital and/or healthcare network utilized a computer network that includes shared bandwidth. The hospital centralizes image reconstruction into one of two locations (e.g. each location includes a scheduling node and reconstruction node). The first location is dedicated to the hospital and the second location is used as backup within the hospital and provided to others within the healthcare network (e.g. two scheduling nodes). The healthcare network contracts with a service provider for a third scheduling and reconstruction location on a per fee basis (third scheduling node). Thus, the scheduling node priority 404 of the imaging location includes in the scheduling nodes ordered by the first location dedicated to the hospital, the second location of the healthcare network computer network, and the service provider as the third node. The hospital network and/or the service provider can include other scheduling and reconstruction nodes in the scheduling node priority list 312, which includes scheduling nodes and reconstruction nodes distributed geographically and used as referral nodes. Alternatively, the scheduling node priority 404 can include a single location, which is the scheduling node dedicated to the hospital, the scheduling node dedicated to the hospital can use the scheduling node priority list 312 to refer to the healthcare network scheduler and/or the service provider.

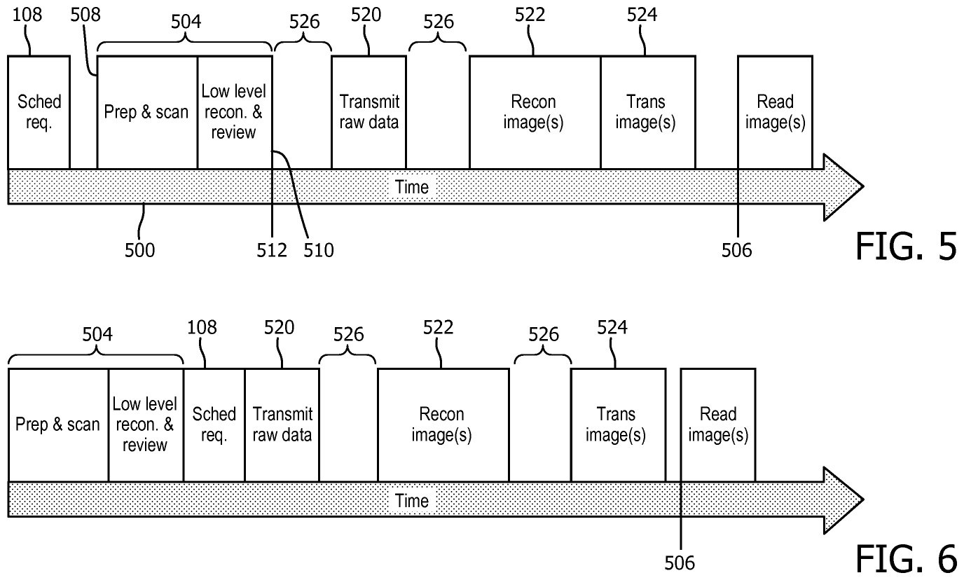

Network bandwidth, cost considerations, and/or management policies may indicate which scheduling nodes appear in either of the scheduling node priority list 312 (and where deployed) and the scheduling node priority 404. Locations of deployment of the medical imaging scanners 112 and associated infrastructure can be in response to healthcare needs indicated by healthcare practitioners. For example, physician input can indicate healthcare aspects served by a particular location of the medical imaging scanner 112. A location that serves primarily cardiac patients can have different scheduling and/or delivery priority requirements than a location direct to rheumatology patients. Similarly, an administrative manager may be aware of different infrastructure constraints between locations, such as imaging support staffing or supporting network infrastructure. For example, network infrastructure of a remote location may support cost effective use of an external service more than installing increased bandwidth to a centralized campus location. FIG. 5 schematically illustrates an embodiment of static scheduling with events, and each event includes a duration, a start time and an end time. The events are illustrated by boxes placed on a horizontal time line 500. The times can include one or more statistical estimates of the event time, such as a mean, medium, maximum, minimum, standard deviation, etc. The reconstruction request 108 that is static is received by the scheduler 302 prior to an imaging examination 504. The reconstruction request 108 is according to an imaging protocol, which can be obtained from the work-list 402, manual entry and/or selection from a list of imaging protocols.

The reconstruction request 108 can include a first time 506, such as a time to read, a time to diagnosis, or a time to plan a treatment. The time to read identifies an expected time of delivery of the reconstructed medical image 118 to the destination node 120, which requested to be satisfied by the scheduling node 104. In some embodiments, the time to read can include a default, such as within 24 hours of the imaging examination, a predetermined time a next business day, a time a next reader is available to read the reconstructed medical image 118 based on work schedules, etc. In some embodiments, the time to delivery is omitted, and a forecast is requested.

The imaging examination 504 can include a time for preparation and scanning 508 of the subject, and time for an initial reconstruction and review 510 by a healthcare practitioner, after which the raw image data 114 is available for transmission at a second time 512.

The scheduler 302 schedules a third time 520 to transmit the raw image data 114 over the network 102 from the source node 116 to the reconstruction node 106, a fourth time 522 to reconstruct the raw image data 114 into the reconstructed medical image 118 by the reconstruction unit 304, and a fifth time 524 to transmit the reconstructed medical image 118 over the network 102 to the destination node 120. The third time 520, fourth time 522, and the fifth time 524 can follow in sequence or each include a deferral or delay 526. For example, transmission of the raw image data 114 can delayed to coincidence with available network bandwidth from the source node 116, e.g. the medical imaging scanner 112 to the scheduling node 104. The reconstruction by the reconstruction unit 304 can be delayed based on the scheduling algorithm.

The scheduler 302 can optimize scheduling of the third time 520, fourth time 522, and the fifth time 524 according to the scheduling algorithm(s). Optimization can include adding the delay 526 before and/or after an event, and can include a reordering of schedule between different reconstruction requests 108. Each delay 526 can be different. For example, the scheduler 302 schedules the following TR_{A}, TR_{B}, RI_{A}, RI_{B}, TI_{A}, TI_{B}, where TR is the transmitting raw image data event, RI is reconstruction event, and TI is transmitting reconstructed image data event for reconstruction requests X and Y respectively. The scheduler 302 can optimize the schedule by reordering the events, such as TR_{A}, TR_{B}, RI_{B}, RI_{A}, TI_{B}, TI_{A} Criteria for optimization can include the time to read 506. In some embodiments the criteria for optimization can include throughput or the delivery priority.

FIG. 6 schematically illustrates an embodiment of dynamic scheduling. The reconstruction request 108, which is dynamic, occurs after the completed imaging examination 504. Such a situation can include changing imaging protocols for the imaging examination based on patient condition, an emergency imaging examination, and the like. For example, a first imaging examination is dynamically scheduled with a change in imaging protocol, and a second imaging examination is dynamically scheduled as an emergency with a higher delivery priority than the first imaging examination (i.e. the second imaging examination is given a higher priority to the scheduling algorithm than the first imaging examination).

The higher delivery priority can be in response to a clinical need, which selects a higher priority scheduling queue of the multi-level queue scheduling algorithm. For example, imaging a chest of a patient experiencing chest pain is given a higher delivery priority than imaging a hand of a patient with a non-life threatening hand injury. The higher delivery priority can be included in the node priority 404 and specify a same or a different ordering of node priorities. For example, a list represented as {delivery priority 1, ordered A, B, {unordered C, D, B} } can represent both a first ordering of nodes and a first selection of a queue of a highest priority, while {delivery priority 2, ordered B, A, {unordered D, B} } represents both a second ordering of nodes and a second selection of a lower priority queue. The delivery priority is represented with an element indicative of a relative priority, e.g. delivery priority X, which can be included with or provided separately from the node priority 404. X can be selected from a set of values, which correspond to queues scheduled with different priorities in a multi-queue scheduling algorithm. When used in combination with the node priorities, the node priority 404 can enforce policies of which nodes are used, which can be associated with certain policies, such as those directed to cost considerations.

The third time 520 to transmit the raw image data 114 over the network 102 from the source node 116 to the reconstruction node 106 can begin immediately following the imaging examination 504 or can start even concurrently. The delays 526 can be added to allow currently busy resources to become available and/or to account for priorities in scheduling.

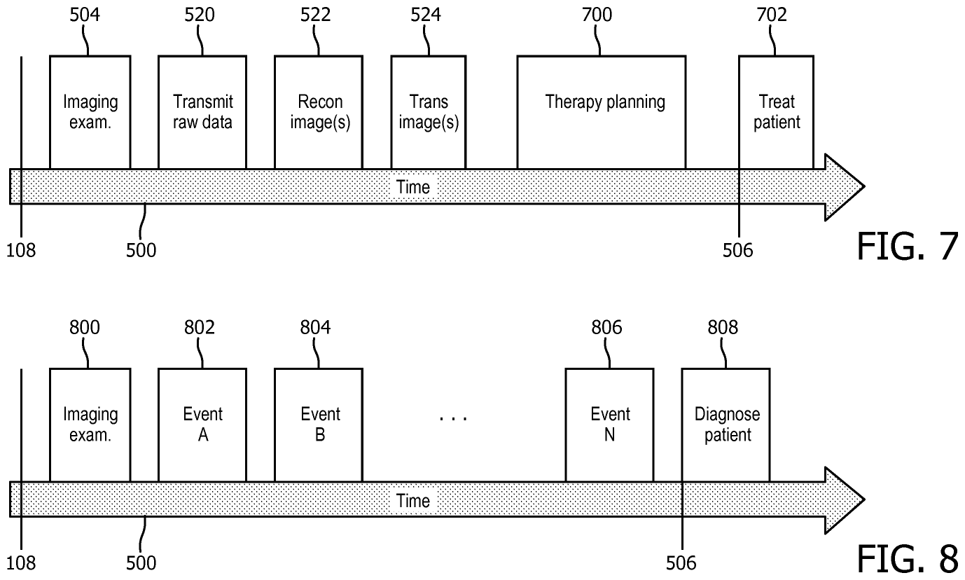

FIG. 7 schematically illustrates an embodiment of static scheduling. The scheduling includes the imaging examination, the third time 520 to transmit the raw image data 114 over the network 102 from the source node 116 to the reconstruction node 106, the fourth time 522 to reconstruct the raw image data 114 into the reconstructed medical image 118 by the reconstruction unit 304, the fifth time 524 to transmit the reconstructed medical image 118 over the network 102 to the destination node 120, such as a therapy planning system (not shown). The scheduling further includes a sixth time 700, which includes planning a therapy for the patient from the reconstructed medical image 118. The planning can include external beam radiation therapy, radiation seed therapy, chemo therapy, combinations thereof and the like. The sixth time 700 delivers a proposed therapy at the delivery time 506, which includes a next appointment with the patient to receive the proposed therapy.

In some embodiments, one of the third time 520 or the fifth time 524 can be omitted. For example, reconstruction hardware can be shared with radiation therapy planning, in which the destination node 122 and the reconstruction node 106 include a common storage subsystem.

FIG. 8 schematically illustrates an embodiment of static scheduling, which includes N events after the imaging examination 504, where N is an integer and greater than or equal to 2. The time to delivery 506 includes a time to diagnose, such as a follow-up appointment with a patient who undergoes an imaging examination, and then returns to an ordering physician for a medical diagnosis based on the imaging examination 504 and information derived from the imaging examination 504. The times for N events can include combinations of the third time 520, the fourth time 522, the fifth time 524, a time of generating views, a time of generating a flypath, a time of segmenting an anatomical structure, a time of fitting an anatomical or systemic model, a time of generating a parametric map, a time of quantitatively measuring a metabolic or anatomic feature, a time of identifying and processing relevant prior medical reports for a same patient, a time of processing relevant medical reports for comparison to a like patient population, a time of therapy planning and/or simulation, a time for reading, etc.

The estimates times for each of the N events can determine or modeled from the history 130. The estimated times or models for each of the N events can be included in the events database 311, from which the scheduler 302 can use to schedule according to the time to delivery 506.

The scheduling can include assignment of resources within one or more of the N events. For example, a time to read includes a qualified healthcare practitioner, such as a radiologist, reviewing the reconstructed medical image 118, and generating a medical report that includes findings, diagnoses, and recommendations. At the time of scheduling the imaging examination 504, the scheduling can include assigning the qualified healthcare practitioner that will read the reconstructed medical image 118 from the scheduled imaging examination 504. The qualified healthcare practitioner can be selected based on a matching of qualifications of the healthcare practitioner and the imaging protocol of the imaging examination. In some embodiments, the qualified healthcare practitioner can be assigned to a pool of qualified healthcare practitioners, such as, for example, qualified to read reconstructed medical images according to imaging protocol A, and the assignment made to the pool.

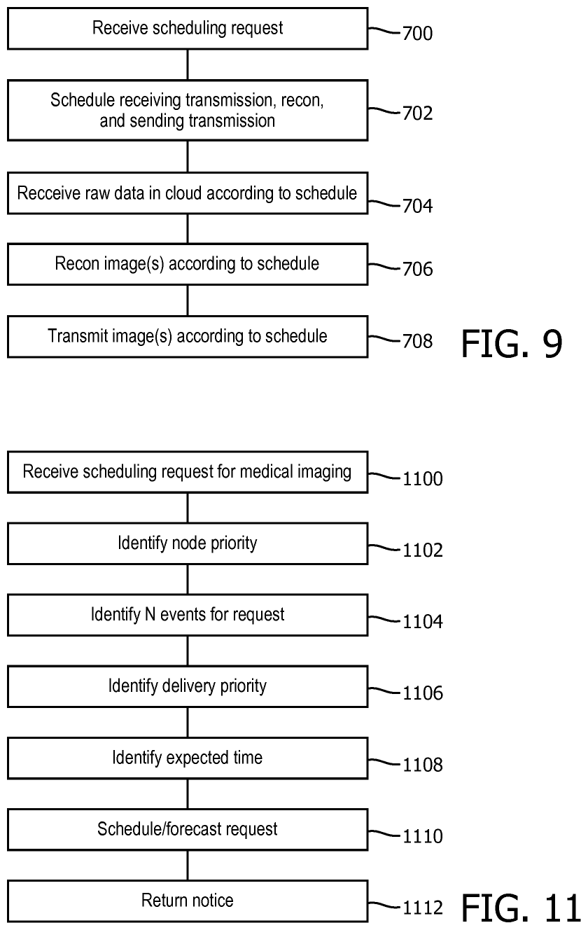

FIG. 9 illustrates a flowchart in accordance with an embodiment(s) herein.

At 900, a reconstruction request 108 according to an imaging protocol is received from a reconstruction requesting node 112. The reconstruction request 108 can include the time to read 506, which identifies a time for completed delivery of the reconstructed medical image 118 at the destination node 120. The reconstruction request 108 can include a scheduled time of an imaging examination. The reconstruction request 108 can include the work-list 402 of the medical imaging scanner 112. The reconstruction request 108 can include the source node 116 and the destination node 120.

At 902, a schedule is generated, which includes a first time to transmit the raw image data 114 over the network 102 from the source node 116, a second time to reconstruct the medical image 118 by the reconstruction node 106, and a third time to transmit the reconstructed medical image 118 over the network 102 to the destination node 120. The generated schedule or a forecast can be returned to the reconstruction requesting node 110. In some embodiments, a notice is returned if the reconstruction request 108 cannot be satisfied by the time to read 506. In some embodiments, the scheduling node priority list 312 can be used to refer the reconstruction request 108 to other scheduling nodes 104.

At 904, the raw image data 114 is transmitted over the network 102 from the source node 116 to the reconstruction node 106 according to the schedule.

At 906, the raw image data 114 is reconstructed into the reconstructed medical image 118 by the reconstruction units 304 of the reconstruction node 106 according to the schedule.

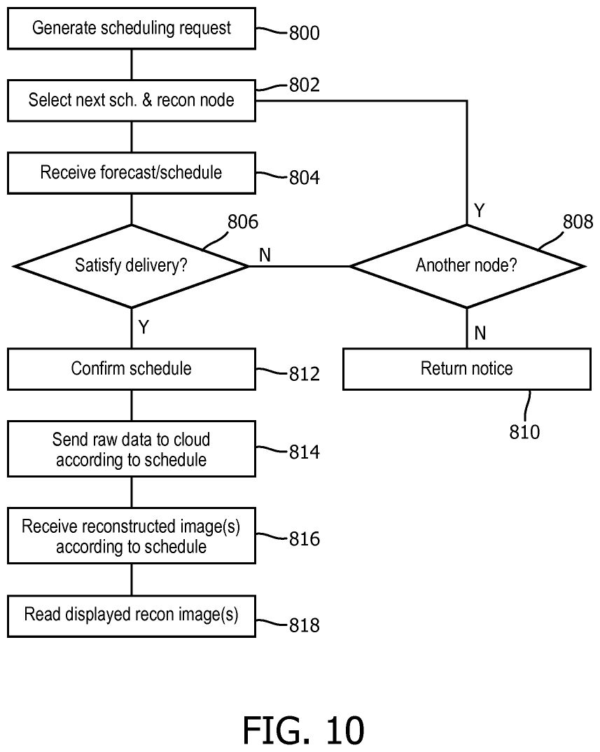

At 908, the reconstructed medical image 118 is transmitted over the network 102 from the reconstruction node 106 to the destination node 120 according to the schedule. FIG. 10 illustrates a flowchart in accordance with an embodiment(s) herein.

At 1000, a reconstruction request 108 according to an imaging protocol is generated.

At 1002, the scheduling node 104 is selected and the reconstruction request 108 is submitted to the scheduling node 104. The scheduler 302 of the scheduling node 104 determines the schedule, which includes a first time to transmit the raw image data 114 over a first network from the source node 116 to the reconstruction node 106, a second time to reconstruct the medical image 118 by the reconstruction node 106, and a third time to transmit the reconstructed medical image 118 over a second network from the reconstruction node 106 to the destination node 120. The selection can include selecting a next scheduling node 104 from the scheduling node priority 404.

At 1004, a forecast or schedule acknowledgement can be received from the selected scheduling node 104, which includes an expected delivery of the reconstructed images 118 at the destination node 120. In some embodiments, acts 802 and 804 are performed in parallel for each scheduling node 104 in the scheduling node priority 404 and include the forecast by the scheduler 302. In some embodiments, schedule acknowledgement includes the schedule by the scheduler 302. In some embodiments, the schedule acknowledgement includes a notice indicative of whether the reconstruction request 108 can be satisfied by the selected scheduling node 104.

At 1006, a decision is made whether a response from the selected scheduling node 104 satisfies delivery requirements. In some embodiments, satisfying delivery is whether the reconstructed image 118 can be delivered to the destination node 120 before the time to read. In some embodiments, the delivery requirements include a selection of one of the forecasts.

At 1008, a decision is made whether additional scheduling nodes 104 in the node priority 404 are to receive the reconstruction request 108 for instances of act 806 where the response includes non-satisfactory delivery. If additional scheduling nodes 104 are present in the node priority 404, then acts 802 and 806 are repeated. If no additional scheduling nodes 104 are present in the node priority 404, then a notice is returned at 810 indicating that no scheduling nodes 104, e.g. in the node priority 404, can satisfy the delivery. In some embodiments, acts 808, 810 are omitted, such as when node priority 404 is omitted.

At 1012, a confirmation of the reconstruction request 108 can be sent to a corresponding scheduling node 104. In some embodiments, the confirmation includes the selection of the scheduling node 104 with a best forecast of delivery. In some embodiments, the confirmation includes the selection of the scheduling node 104 that first satisfies delivery in the time to read. In some embodiments, the act is omitted.

At 1014, the raw image data 114 is transmitted or sent from the source node 116 to the reconstruction node 106 according to the schedule.

At 1016, the reconstructed medical image 118 is transmitted from the reconstruction node 106 to the destination node 120 according to the schedule.

At 1018, the reconstructed medical image 118 is read displayed on a display device.

FIG. 11 illustrates a flowchart in accordance with an embodiment(s) herein.

At 1100, the configured processor 330 of the scheduler node 104 receives the scheduling request 108 for the medical imaging examination 504 of a subject. The scheduling request 108 can include the time to delivery 506 or a request for a forecast. The scheduling request 108 includes the imaging protocol or information from which the imaging protocol can be identified, such as modality and anatomy. The scheduling request 108 can include information indicating other events subsequent to the imaging examination, such as post processing instructions or therapy planning. The scheduling request 108 can include the delivery priority. The scheduling request 108 can include information from the node priority 404.

At 1102, the configured processor 330 identifies the node priority for the received scheduling request 108, which indicate the source node, the destination node, and an order of reconstruction nodes, and resources of each node available to satisfy the received scheduling request 108. The resources include transmission resources between nodes. In some embodiments, the node priority can include nodes from the node priority 312, the node priority 404, and combinations thereof. In some embodiments, the node priority defaults to one reconstruction node associated with the scheduler node 104.

At 1104, the configured processor 330 identifies the N events that satisfy the received scheduling request 108 based on the imaging protocol. The N events include the medical imaging examination 504 and at least one other event. The other events can include the transmitting the raw data to the reconstruction node 106, transmitting the reconstructed medical image 118 to the destination node 120, planning a therapy based on the reconstructed medical image 118, post processing of the reconstructed medical image 118, reading the reconstructed medical image 118, and combinations thereof. The identification of the N events includes determining scheduling parameters for each event from the imaging protocol database 310, the transmission resource database 309, the event database 311, and combinations thereof.

At 1106, the configured processor 330 can identify the delivery priority. The delivery priority can include a relative priority to other scheduling requests. For example, a first scheduling request includes a delivery priority of “1” or “stat” according to a clinical need, while a second delivery priority of “2” or “normal” processing. The configured processor 330 associates the identified delivery priority with service estimates of each event. In some embodiments, the delivery priority can be identified from the node priority 404. In some embodiments, the delivery priority can be omitted.

At 1108, the configured processor 330 can identify the delivery time 506. In some embodiments, a forecast request is alternatively identified.

At 1110, the configured processor 330 schedules the received scheduling request according to the identified N events, estimates for each event, and the identified delivery time 506. The scheduling can include a selection of one reconstruction node from a plurality of reconstruction nodes of the identified node priority. The scheduling can include a selection of estimates for each event according to the delivery priority. Alternatively, the configured processor 330 forecasts the received scheduling request according to the identified N events and estimates for each event.

At 1112, the configured processor 330 returns a forecast or schedule acknowledgement to the reconstruction requesting node 110. The acknowledgement can include the schedule of the N events according to the delivery time 506. The acknowledgement can include a forecast according to an earliest delivery time of the N events.

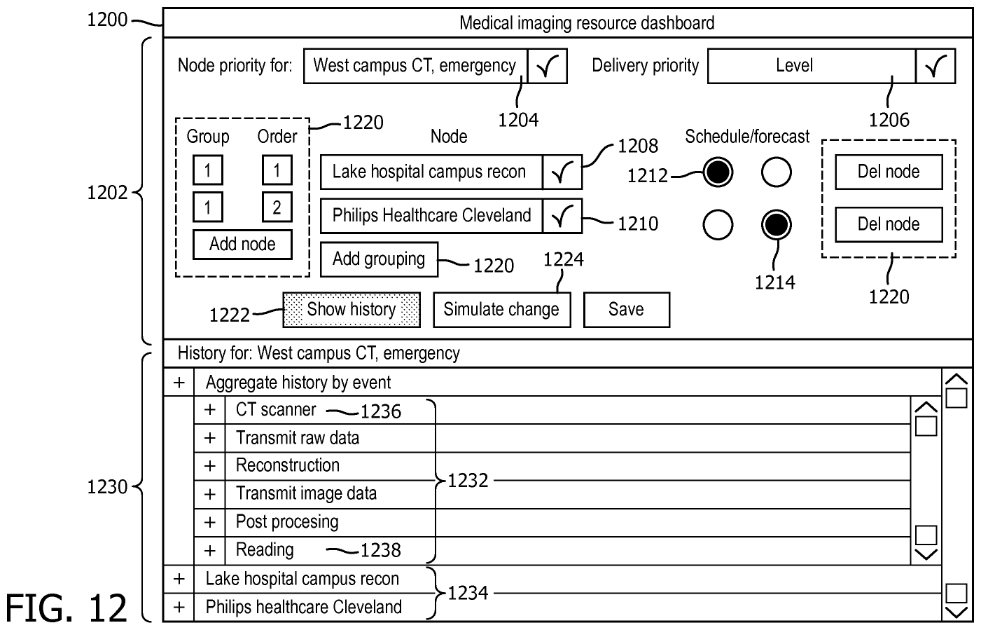

FIG. 12 depicts an example dashboard display 1200 generated by the dashboard 132. The dashboard display 1200 can include a first region 1202, which allows a user to add, display, and/or modify a node priority configuration, such as the node priority 404 or the node priority 312. For example, the node priority is illustrated for “West Campus CT, Emergency” 1204. The node priority configuration includes the delivery priority of “Level 1” 1206, which can be modified.

The first region 1202 includes two displayed and ordered reconstruction nodes. The first reconstruction node is “Lake Hospital Campus Recon” 1208, and the second ordered reconstruction node is “Philips Healthcare Cleveland” 1210. The dashboard display 1200 includes an indicator 1212 that requests from “West Campus CT, Emergency” 1204 are first attempted by the scheduler node 104 to schedule with the reconstruction node represented by “Lake Hospital Campus Recon” according to the delivery time in each request. If the request cannot be satisfied according to the delivery time by the first reconstruction node of “Lake Hospital Campus Recon” 1208, then a second indicator 1214 indicates the request is submitted to the second reconstruction node of “Philips Healthcare Cleveland” 1210 for a forecast.

The dashboard display 1200 can include controls 1220 that add, delete, reorder, or regroup reconstruction nodes 106 in the configured node priority.

The dashboard display 1200 can include a history indicator 1222 to display history from the history 130 in a second region 1230 according to the configured node priority. The displayed history can be aggregated for all requests by event 1232 or detailed further by node for each event 1234 in the configured node priority. For example, a “CT scanner” event 1236 can be expanded (e.g. mouse click on “+sign) and statistics of throughput, resource utilization, actual time versus scheduled time to delivery, etc., can be displayed. The display for each event can include statements of assigned resources or resource estimates from the imaging protocol database 310, the transmission resource database 309, or the event database 311. The display can include descriptive statistics of the history 130 according to the event. The display can include further breakdowns, such by date, time, day of week, etc. For example, the display can include a maximum number of readers of an ith event, which is reading the reconstructed image and generating a radiological report. The statistics can include a mean, a maximum and a minimum utilization of the readers, such as a maximum of 24 readers were utilized on Tuesday of 28 available readers, and on Wednesday 23 readers averaged 6.5 hours reading with a minimum time of 0.8 hours and for the week the day with the least number of readers utilized was Thursday with 16. The display can include graphical displays of the information, such as bar charts, pie charts, line graphs, etc. The display can include connections to other systems, such as a work scheduling system for readers.

For example, review of an expanded view of the “Reading” display 1238 can include displaying reader utilization, and connection to a scheduling system for requesting additional readers. Thus, an instance where readers of virtual colonoscopies currently includes two readers who are assigned and utilized 100%, suggests a third reader may need to be assigned.

The dashboard display 1200 can include a simulator indicator 1224, which displays a simulated effect of a change in the configured node priority, a change of resources in one or more of the events, a change in composition of the n-events, and combinations thereof. The simulated effect can be presented alternately or in addition the displayed history in the second region 230. The processor 330, 420 performs the simulation using the history 130 and the entered combination of changes in configured node priority, changes in resources, and changes in composition of the n-events.

For example, the history 130 includes operation of the system 100 using a reconstruction node R, and a simulation simulates adding a second reconstruction node R′. The second reconstruction node R′ is added using the first region 1202 of the dashboard display 1200 and the simulator indicator 1224 is selected using an input from the input device 324, 414. The processor 330, 420 simulates the combined use of reconstruction nodes R and R′ using the history 130, and displays the result of the simulation in the second region 1230. The displayed simulation results can include descriptive statistics or graphs comparing the simulated results of the combined reconstruction nodes R and R′ with the actual history of reconstruction node R. For example, the statistics of average and maximum utilization of the combined reconstruction nodes R and R′ can be compared numerically or graphically with average and maximum utilization of R. The descriptive statistics, such as average, minimum and maximum of the time to read for the combined reconstruction nodes R and R′ can be compared with the same statistics of the actual history of the time to read for the reconstruction node R.

The statistics can include statistics based on time for each event or combined events, resource quantities for each event, cost assignments for each resource or event, or combinations thereof. For example, the statistics of time can be based on time to be processed at each event, or time for processing to a point of delivery, such as time to read, time to diagnose, time to treatment, etc. The time can be assigned a cost or price value, and the statistics expressed in units of dollars instead of time for financial comparison. The statistics of resource quantities can be based on resources assigned to an event. For example, a reconstruction node Z include 4 reconstruction units, and the statistics include an average and a maximum use of the 4 reconstruction units. Each of the reconstructions can be assigned a price, such as in a service model, and each of the reconstruction units can be assigned a cost. The statistics can include a total price of the reconstructions and a total cost of the 4 reconstruction units.

For example, the simulation can provide an estimate of additional reconstruction units to be added, such as within a hospital network or purchased as time chunks from a third party. Resource management can include estimates of additional reconstruction units, network bandwidth, additional readers, and the like. Simulations can include changing schedule of an event and measuring the impact on a subsequent event. For example, changing 10% of transmission time to delay until after midnight can gain a 20% improvement in reading time and completion of a time to diagnose. The simulations can be utilized to optimize imaging examination scheduling and transmission times.

In another example, an ith event includes readers that are qualified healthcare practitioners reading the reconstructed images. The history 130 includes times for reading reconstructed images according to the imaging protocol for each reader. Simulations can include changing the mix of readers, such as adding or removing readers. For example, a new reader can be added to the resources for reading the reconstructed images and assigned a profile based on estimated times for reading reconstructed images. The simulation applies the history 130 of reading images to assigned readers, which simulates adding the new reader to the assigned group qualified for protocol X. The estimated times for reading reconstructed images can be based on the history 130 from other readers, such as those likewise qualified.

Simulations can include changing the types of reconstructed images read by one or more readers based on the protocol. For example, the protocols of reconstructed images can be changed for a reader and a simulation of the effect performed, such as, reader X previously read reconstructed images according to protocols A, B and C, and a change simulates reading reconstructed images according to protocols A and B, omitting C, and adding D. The simulation includes using the history 130 of the readers assigned to the ith event over selected protocols A, B, C and D or over all protocols.

In some embodiments, the simulator indicator 1224, can simulate use of alternative resources for an ith event, or simulate use of an alternative protocol. For example, a reconstruction node 106 includes three reconstruction units 304 of A, B, and C and the ith event is reconstruction. A candidate replacement unit or upgrade for unit B is B′, which can perform the same reconstructions of B in 40% of the time. The simulator indicator 1224 can include simulating the effect on one or all protocols based on substituting the reconstruction unit B′ for reconstruction unit B. That is, using the history 130 and substituting the performance characteristics of B′ for B.

The simulation can include combinations changing the composition of events for a protocol. For example, an entity considers changing a scanner, which eliminates a post processing event for protocol D, and can perform the imaging examination for protocol E in 60% of a time of a current scanner. The simulation includes using the history 130 for a protocol D′, which includes the history of D with the post processing event omitted, and reducing times, t from the history of the imaging examination 504 for protocol E to 0.6*t of the history.

The simulations can help to optimize scheduling and operational efficiency. New events, changed events according to equipment replacement, changed events according to equipment upgrades, changed events according to start times, changed events according to reader scheduled, omitted events, and combinations thereof can be simulated to determine overall impact, such as a time to diagnose, time to read, time to reimbursement, etc. That is, with different inputs, such as a duration of the read time, transmission time, and capital equipment items, the simulation and dashboard display 1200 can identify operational efficiencies, optimize scheduling, and provide views of tradeoffs between the improvements and cost to achieve the improvements. Combining the simulations with the cost/price information, the simulations can provide financial forecasting and optimization through views in the dashboard display 1200 that include time improvements, cost reductions, patient wait times, time to diagnose, time to reimbursement, etc.

The above may be implemented by way of computer readable instructions, encoded or embedded on a computer readable storage medium, which, when executed by a computer processor(s), cause the processor(s) to carry out the described acts. Additionally or alternatively, at least one of the computer readable instructions is carried out by a signal, carrier wave or other transitory medium.

The above steps can be performed in a different order and/or some steps can be omitted.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage.

A computer program may be stored/distributed on a suitable medium, such as an optical storage medium or a solid-state medium supplied together with, or as part of other hardware, but may also be distributed in other forms, such as via the Internet or other wired or wireless telecommunication systems. Any reference signs in the claims should not be construed as limiting the scope.

1. A system for reconstruction of medical images over a network, comprising:
a digital storage memory configured to store processor executable instructions; and
at least one processor configured to execute the processor executable instructions to schedule a reconstruction request, wherein the reconstruction request includes a medical image reconstruction of a subject according to an imaging protocol, wherein the imaging protocol is mappable to a resource requirement of at least one of transmission of raw image data from a source node to a reconstruction node, timing and duration of reconstruction at the reconstruction node, and timing and duration of transmission of the reconstructed medical image data from the reconstruction node to a destination node, and the scheduling comprises scheduling of a plurality of events, each event with a corresponding time, and the plurality of events include at least one event with the corresponding time selected from at least one of a first time to transmit the raw image data over a first network from the source node to the reconstruction node, a second time to reconstruct the medical image by the reconstruction node, and a third time to transmit the reconstructed medical image over a second network from the reconstruction node to the destination node.

2. The system according to claim 1, wherein the at least one processor is further configured to schedule a plurality of reconstruction requests, each from a different source node.

3. The system according to claim 1, wherein at least one time includes a delay, and the at least one time is selected from at least one of the first time, the second time, and the third time.

4. The system according to claim 1, wherein the at least one processor is further configured to return a forecast that comprises an expected completion time of delivery to the destination node.

5. The system according to claim 1, wherein the at least one processor is further configured to schedule the reconstruction request according to a time to read for each reconstruction request.

6. The system according to claim 1, wherein the reconstruction request is received prior to an imaging examination according to the imaging protocol, and the reconstruction request further comprises a fourth time of the imaging examination.

7. The system according to claim 1, wherein the reconstruction request is received after an imaging examination according to the imaging protocol, and the reconstruction request includes a delivery priority.

8. The system according to claim 1, wherein the raw imaging data is generated from a medical imaging scanner with at least one modality selected from one of Computed Tomography, Magnetic Resonance, Positron Emission Tomography, Single Proton Emission Computed Tomography, X-ray and Ultrasound.

9. The system according to claim 1, wherein the scheduling of the plurality of events includes scheduling according to a delivery time, wherein one of the plurality of events includes the second time to reconstruct the medical image by the reconstruction node.

10-19. (canceled)

20. A method for reconstructing medical images over a network, comprising:
scheduling a reconstruction request for a medical image reconstruction of a subject according to an imaging protocol, wherein the imaging protocol is mappable to a resource requirement of at least one of transmission of raw image data from a source node to a reconstruction node, timing and duration of reconstruction at the reconstruction node, and timing and duration of transmission of the reconstructed medical image data from the reconstruction node to a destination node, wherein the scheduling comprises scheduling of a plurality of events, each event with a corresponding time, and the plurality of events include at least one event with the corresponding time selected from at least one of a first time to transmit raw image data over a first network from a source node to a reconstruction node, a second time to reconstruct the medical image by the reconstruction node, and a third time to transmit the reconstructed medical image over a second network from the reconstruction node to a destination node.

21. The method according to claim 20, wherein scheduling comprises scheduling a plurality of reconstruction requests, each reconstruction request includes a different source node.

22-25. (canceled)

26. A non-transitory computer-readable storage medium having executable instructions stored thereon which, when executed by at least one processor, cause the at least one processor to perform a method for reconstructing medical images over a network, the method comprising:
scheduling a reconstruction request for a medical image reconstruction of a subject according to an imaging protocol, wherein the imaging protocol is mappable to a resource requirement of at least one of transmission of raw image data from a source node to a reconstruction node, timing and duration of reconstruction at the reconstruction node, and timing and duration of transmission of the reconstructed medical image data from the reconstruction node to a destination node, wherein the scheduling comprises scheduling of a plurality of events, each event with a corresponding time, and the plurality of events include at least one event with the corresponding time selected from a group consisting of a first time to transmit raw image data over a first network from a source node to a reconstruction node, a second time to reconstruct the medical image by the reconstruction node, and a third time to transmit the reconstructed medical image over a second network from the reconstruction node to a destination node.

27-50. (canceled)
